(19) United States
(12) Patent Application Publication
(10) Pub. No.: US 2023/0313182 A1
(43) Pub. Date: Oct. 5, 2023

(54) PREPARATION METHOD FOR DIABETES EARLY WARNING AND/OR DIAGNOSTIC REAGENT KIT BASED ON HSA-MIR-320A, MEDICAMENT FOR PREVENTING DIABETES, SCREENING METHOD FOR MEDICAMENT, AND PREPARATION METHOD THEREFOR

(71) Applicant: Tongji Hospital, Tongji Medical College, Huazhong University of Science and Technology, Wuhan (CN)

(72) Inventors: Daowen WANG, Wuhan (CN); Chen CHEN, Wuhan (CN); Huaping LI, Wuhan (CN); Feng WANG, Wuhan (CN); Zhongwei YIN, Wuhan (CN)

(21) Appl. No.: 17/904,597

(22) PCT Filed: May 18, 2020
(86) PCT No.: PCT/CN2020/090794

§ 371 Date: Aug. 19, 2022

(30) Foreign Application Priority Data
Jun. 11, 2019 (CN) 201910500477.9
Jun. 11, 2019 (CN) 201910500630.8

(51) Int. Cl. C12N 15/113; C12N 15/86; C12Q 1/6883; A61P 3/10

(52) U.S. Cl. CPC C12N 15/113; C12N 15/86; C12N 2310/113; A61P 3/10; C12Q 2600/178; C12Q 1/6883

(57) ABSTRACT

The present invention, “a medicament for preventing and treating diabetes based on has-miR-320a, a screening method for the medicament, and a preparation method therefor,” relates to the field of molecular medicine and the diagnosis, prevention, and treatment of metabolic diseases. The active ingredient of the medicament has has-miR-320a as the medicinal target and expresses the has-miR-320a by means of bonding, and/or capturing, and/or degrading, and/or reducing to provide the efficacy of diabetes prevention. The has-miR-320a is as represented by SEQ ID NO: 1. Moreover, also provided in the present invention are the screening method and the preparation method for the medicament. The employment of the medicament of the present invention significantly reduces the blood sugar concentration of a diabetic, effectively alleviates the symptoms of diabetes complications of a test animal, and allows the test animal to maintain healthy and stable blood sugar over a long time.

### CROSS-REFERENCE TO RELATED APPLICATIONS

This application is a National Stage Appl. filed under 35 USC 371 of International Patent Application No. PCT/CN2020/090794 with an international filing date of May 18, 2020, designating the United States, now pending, and further claims foreign priority benefits to Chinese Patent Application No. 201910500477.9 filed Jun. 11, 2019, and to Chinese Patent Application No. 201910500630.8 filed Jun. 11, 2019. Inquiries from the public to applicants or assignees concerning this document or the related applications should be directed to: Matthias Scholl P. C., Attn.: Dr. Matthias Scholl Esq., 245 First Street, 18th Floor, and Cambridge, MA 02142.

### TECHNICAL FIELD

The disclosure relates to field of molecular medicine and diagnosis, prevention and treatment of metabolic diseases, and more particularly to a method for preparing a kit for early warning and/or diagnosing diabetes, and a drug for preventing and treating diabetes based on hsa-miR-320a and its screening method as well as preparation method thereof.

### BACKGROUND

MicroRNA (miRNA, miR) is a newly discovered class of endogenous small non-coding single stranded RNA with a length about 22 nucleotides which is from endogenous hairpin structure transcript. MicroRNA regulate gene expression after transcription by specifically binding to 3′untranslated region (3′untranslated region, 3′UTR) of target mRNA to promote degradation of target mRNA or inhibit translation of target gene and reduce coding protein level. miRNA is associated with various diseases, including neurodegenerative diseases, heart diseases, kidney diseases and tumor, etc. Recent studies have found that miRNA plays an important role in regulation of glucose and lipid metabolism and insulin resistance.

Diabetes mellitus (DM) is a major disease that seriously threatens human life and health, and its prevalence rate is about 4.4% to 17.9% globally at present, which brings a heavy burden to patients and society. According to epidemiological statistics, the number of diabetes patients in the world reached 370 million in 2011, 80% of which were in developing countries. A total of 4.6 million people died of diabetes in the world that year, and the medical cost of diabetes in the world of that year reached 465 billion US dollars. In China, prevalence rate of diabetes has increased significantly in past 30 years. In 2013, the latest epidemiological survey warned that prevalence rate of diabetes among Chinese adults was 11.6% (about 110 million), and prevalence rate of prediabetes was as high as 50.1% (about 490 million).

More than 90% of diabetes patients are type 2 diabetes mellitus, which is a severe disorder of glycolipid metabolism caused by insulin resistance and p-cell failure and eventually leads to heart cerebrovascular and kidney complications and even death. Previous studies have shown that insulin resistance is a disorder of glycolipid metabolism caused by interaction of various genetic and environmental factors, including defective insulin signaling pathway, abnormal expression of insulin target and cross action of other hormone systems, and imbalance of other metabolic pathways. However, its specific pathophysiological mechanism and molecular signaling network are far from being fully understood and elucidated. Previously, pharmacotherapy strategies for diabetes have been mainly based on correcting glycolipid metabolism disorders, including drugs that promote insulin secretion (sulfonylureas, glinides, DPP-4 inhibitors) and hypoglycemic drugs through other mechanisms (biguanides, TZDs, α-glycosidase inhibitors). Even if patients receive treatment including insulin injection, only 40% of patients have good blood glucose control, and the metabolic disorders caused by diabetes cannot be effectively corrected. Therefore, new treatment measures are urgently needed. On the other hand, etiology and risk factors of diabetes have not been fully elucidated, so it is necessary to find new risk factors and adopt more effective risk assessment methods.

As an important endogenous regulatory factor, miRNAs are widely involved in regulation of signal transduction pathways in animals and plants. Although regulatory mechanism of miRNA has not been fully elucidated, existing research data indicate that miRNA plays an important role in regulation of glycolipid metabolism, which can not only serve as a new diagnostic marker of diabetes, but also participate in regulation of occurrence and development of diabetic insulin resistance and its complications. A series of studies have found significant differences in miRNA expression profiles in peripheral blood of diabetic patients compared with control population. miRNA-375 is specifically expressed in langerhans' islet β cells and participates in regulating secretion of insulin through the PI3K/PDK1/PKB signal pathway, while miR-375 knockout mice showed a glycolipid metabolism disorder phenotype. High systemic or islet specific let-7b expression results in impaired glucose tolerance and reduced insulin secretion. miR-503 expression is increased in endothelial cells under high glucose and is involved in regulation of diabetic vascular function through mediated by Cdc25A and CCNE1.

RNA interference technology is a promising biotechnology with breakthrough clinical application. In just a few years from its inception, several products have entered clinical trials and achieved great success. As disclosed in Chinese invention patent 201210069179.7, in previous research work, applicant of the invention found that miR-320a has a predictive role in atherosclerotic diseases, and studied to confirm the therapeutic role of anti-miR-320a, an antisense sequence of miR-320a, in atherosclerotic diseases. However, relationship between miR-320a and diabetes has not been found in this field, and the existing technologies lack effective products or drugs in molecular diagnosis and molecular therapy of diabetes.

### SUMMARY

Based on above gaps and requirements in this field, the disclosure further confirms the role of miR-320a in risk prediction of diabetes disease based on original basic work, the role of miR-320a participating in regulation of glucose metabolism and development of diabetes, and the therapeutic role of anti-miR-320a in diabetes. The disclosure provides a novel medicinal use of endogenous non-coding small RNA, more specifically involving microRNA-320a (hsa-miR-320a) and its antisense nucleotide sequence hsa-anti-miR-320a in risk evaluation, prevention and treatment of diabetes mellitus. The invention also relates to the construction of a recombinant adeno-associated virus recombinant (rAAV-miR-320a/rAAV-anti-miR-320a) and its preparation method thereof. More specifically, it's related to cloning of hsa-miR-320a and anti-sense sequence anti-hsa-miR-320a and packaging and preparation of recombinant adeno-associated virus containing hsa-miR-320a and anti-hsa-miR-320a, respectively, and use of the recombinant adeno-associated virus in pharmaco.

The technical solution of the disclosure is as follows:

On the one hand, the disclosure provides a biomarker for the diagnosing and/or early warning diabetes. The biomarker comprises a sequence fragment containing hsa-miR-320a; the hsa-miR-320a is shown as SEQ ID NO: 1.

The biomarker comprises the hsa-miR-320a.

The biomarker is the hsa-miR-320a.

In the 2^{nd} aspect, this invention provides a kit for diagnosing and/or early warning diabetes; the kit comprises a reagent for quantitative detection of the biomarker.

The kit comprises a reagent for quantitative detection of hsa-miR-320a.

The reagent for quantitative detection of hsa-miR-320a comprises a specific primer pairs for hsa-miR-320a.

The specific primer pairs are commercially-available MIRQ0000510-1-1. The primer product is purchased from Guangzhou Ribo Biotechnology Company Limited.

The regents for quantitative detection of hsa-miR-320a also comprises reverse transcription reagents; and/or reverse transcription PCR reagents;

preferably, the reverse transcription reagent comprises: RT Primer Mix, 2×TS reaction buffer, RNase free H₂O, TS enzyme;

further preferably, the reverse transcription PCR reagent comprises: 2×SYBR Green Mix, RNase free H₂O.

The quantitative detection refers to fluorescent quantitative PCR detection;

most preferably, the kit comprises miR-320a RT-primers, miR-320a real-time PCR forward primers, miR-320a real-time PCR reverse primers, SYBR Green I, TS reaction enzyme, TS reaction buffer and DEPC ddH₂O.

In the 3^{rd} aspect, this invention provides Use of the biomarker in preparing reagent for diagnosing diabetes.

In the 4^{th} aspect, this invention provides a drug for preventing and treating diabetes; an active pharmaceutical ingredient of the drug is taking hsa-miR-320a as drug target, and efficacy of preventing and treating diabetes is achieved by binding, and/or capturing, and/or degrading hsa-miR-320a, and/or down-regulating expression of hsa-miR-320a; The hsa-miR-320a is shown as SEQ ID NO: 1.

The active pharmaceutical ingredient of the drug comprises: a substance that can degrade hsa-miR-320a and/or down-regulate expression of hsa-miR-320a;

preferably, the active pharmaceutical ingredient of the drug is a substance that can down-regulate expression of the hsa-miR-320a.

The active pharmaceutical ingredient of the drug comprises: a sequence fragment anti-hsa-miR-320a that is antisense complementary to hsa-miR-320a;

preferably, the antisense complementary refers to that length of the anti-hsa-miR-320a reverse complementary to full-length or partial sequence of the hsa-miR-320a is a sequence fragment of 15-25 bases.

The active pharmaceutical ingredient of the drug is a recombinant plasmid expressing the sequence fragment anti-hsa-miR-320a that is antisense complementary to hsa-miR-320a.

The active pharmaceutical ingredient of the drug is a recombinant adeno-associated virus plasmid pAAV-D(+)-anti-hsa-miR-320a, by which expressed sequence fragment anti-hsa-miR-320a is antisense complementary to hsa-miR-320a;

more specifically, the pAAV-D(+)-anti-miR-320a is constructed by inserting fragment amplified through primer pairs with sequences shown as SEQ ID NO: 4 and SEQ ID NO: 5 into an adenovirus expression vector pAAV-D(+).

The drug also comprises pharmaceutically acceptable excipients and/or reagents for buffering, culturing and/or amplifying the recombinant adeno-associated virus plasmid pAAV-D(+)-anti-miR-320a.

In the 5^{th} of aspect, this invention provides a method of screening drug for preventing and treating diabetes, characterized in that, detecting whether candidate substance can bind, capture and degrade hsa-miR-320a and/or down regulate expression;

preferably, substance that can reduce expression level of hsa-miR-320a is screened.

In the 6^{th} of aspect, this invention provides a method for preparing a drug for preventing and treating diabetes, characterized in that, comprises: taking substance that degrade hsa-miR-320a or down-regulate expression of hsa-miR-320a; sequence fragment anti-hsa-miR-320a antisense complementary to hsa-miR-320a;
recombinant plasmid expressing a sequence fragment of anti-hsa-miR-320a antisense complementary to hsa-miR-320a; and/or,
a recombinant adeno-associated virus vector expressing a sequence fragment of anti-hsa-miR-320a antisense complementary to hsa-miR-320a as an active ingredient in antidiabetic drug.

The method also comprises: primer pairs expressing sequence fragment anti-hsa-miR-320a antisense complementary to hsa-miR-320a is inserted into expression vector to prepare recombinant plasmid stably expressing sequence fragment anti-hsa-miR-320a antisense complementary to hsa-miR-320a.

Primer pairs which can express the sequence fragment of anti-hsa-miR-320a antisense complementary to hsa-miR-320a are shown as SEQ ID NO: 4 and SEQ ID NO: 5;
the expression vector is adeno-associated virus expression vector pAAV-D(+).

Inventor of this invention found by a lot of experiments that expression of hsa-miR-320a in peripheral blood of diabetic patients was significantly increased. Furthermore, the inventor designed and synthesized sequences that express hsa-miR-320a and are antisense complementary to anti-hsa-miR-320a respectively according to hsa-miR-320a base sequence. The sequences are respectively inserted into eukaryotic expression vector pAAV-D (+) to successfully construct a recombinant plasmids pAAV-D (+)-miR-320a and pAAV-D (+)-anti-miR-320a. After that, the following three plasmids were added: 1) pXX2, pXX8 or pXX9, 2) phelper, 3) pAAV-D(+)-miR-320a or pAAV-D(+)-anti-miR-320a were transformed into 293 cells by calcium and phosphorus co-transfection method to package and prepare three serotypes of recombinant adeno-associated viruses (rAAV2, rAAV8 and rAAV9) expressing hsa-miR-320a and antisense complementary anti-hsa-miR-320a respectively. After purification, titer was determined by real-time PCR. Next, two packaged recombinant adeno-associated viruses of same serotype (rAAV-miR-320a and rAAV-anti-miR-320a) were injected into db/db mice via tail vein injection respectively. It was found that blood glucose was significantly regulated in db/db mice, and recombinant adeno-associated virus mediated expression of anti-hsa-miR-320a significantly improved elevated blood glucose and cardiac function in db/db mice. While hsa-miR-320a further aggravated hyperglycemia and heart function in db/db mice. These results further support the therapeutic effect of anti-hsa-miR-320a on diabetes mellitus and its complications.

Specifically, the 1^{st} purpose of the disclosure is to provide expression of miR-320a in peripheral blood of diabetic patients. The results show that expression of miR-320a in peripheral blood of diabetic patients was significantly increased, which suggests that miR-320a can be used as a biomarker for early diagnosing and predicting diabetes.

The second purpose of the disclosure is to confirm the protective effect of anti-miR-320a on diabetes through experiments based on the first purpose, anti-miR-320a can be used as a medicine to treat diabetes and its complications.

In conclusion, hsa-miR-320a discovered in the disclosure can be used as an effective biomarker to evaluate risk of diabetes and to diagnose diabetes. The hsa-miR-320a-based kit is a highly efficient and accurate product in field of diabetes diagnosis. The drug for preventing and treating diabetes based on hsa-miR-320a in the disclosure provides an effective and safe drug for treating diabetes in field of molecular biology, and provides a new choice to treat diabetes for patients and medical staffs.

### DETAILED DESCRIPTION OF THE DRAWINGS

Above and other purposes and features of the invention will become clear from the description given below in conjunction with drawings.

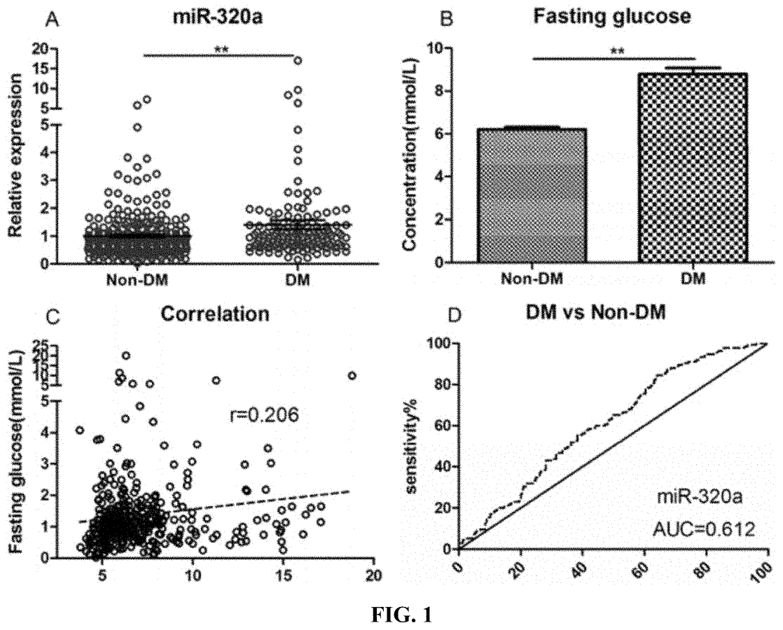

FIG. 1 shows correlation between blood glucose level and hsa-miR-320a expression level in peripheral blood of diabetic patients. A. Expression of hsa-miR-320a in peripheral blood of diabetic patients was detected by real-time PCR; U6 was used as internal reference; method of 2^{-ΔΔ}^{^{CT}} is adopted as comparison method. B. value of fasting blood glucose in patients with diabetes. C. Correlation analysis of hsa-miR-320a expression and fasting blood glucose in peripheral blood. D. ROC curve of peripheral blood hsa-miR-320a for the diagnosis of diabetes mellitus; wherein, **P<0.01; as compared with the control group (n=200). DM: diabetic patients; Non-DM: normal control people.

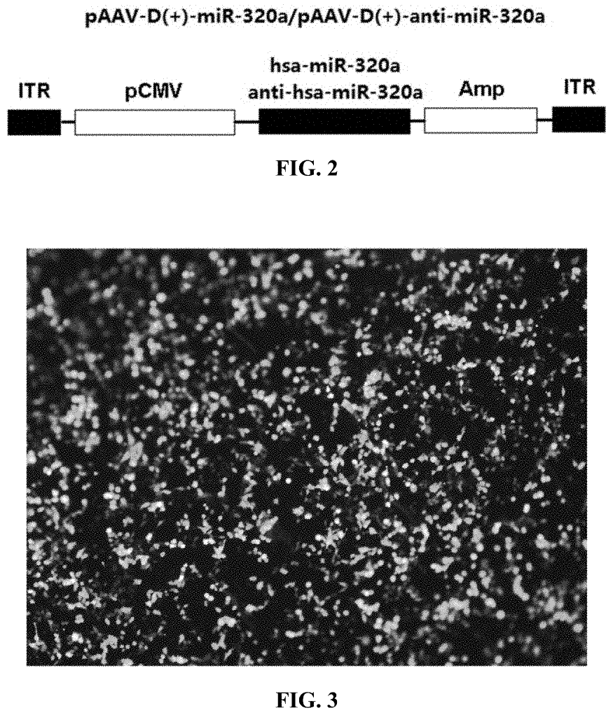

FIG. 2 shows constitution of plasmids of pAAV-D(+)-miR-320a and pAAV-D(+)-anti-miR-320a.

FIG. 3 shows that GFP was observed by fluorescence microscope (40×) after cell transfected by purified virus, the cells that were successfully transfected are shown in green with transfection efficiency more than 90%.

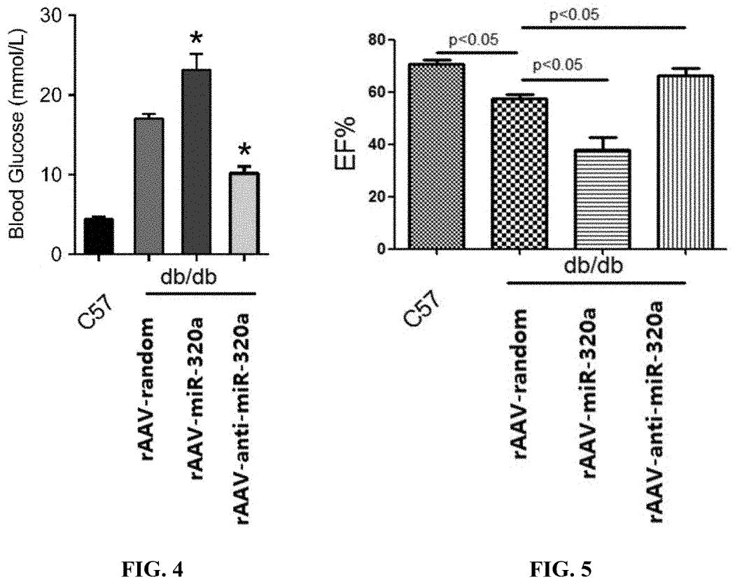

FIG. 4 shows effects of microRNA treatments on fasting blood glucose in db/db mice. It's shown by fasting blood glucose detection that rAAV-miR-320a and rAAV-anti-miR-320a could significantly regulate the glycometabolism of db/db mice. (the former promotes increase of blood glucose, and the latter reduces blood glucose).

FIG. 5 shows effects of microRNA treatments on cardiac ejection fraction in db/db mice. rAAV-anti-miR-320a significantly improve cardiac ejection fraction while rAAV-miR-320a reduces cardiac ejection fraction in db/db mice.

### DETAILED DESCRIPTION

The disclosure will be further illustrated with the following specific examples. Advantages and characteristics of the disclosure will become clearer with the description; however, these embodiments are only exemplary and do not limit the scope of the disclosure. A person skilled in the art should understand that details and forms of the technical solution of the disclosure may be modified or replaced without deviating from the conception and scope of the disclosure, but such modifications and replacements fall within the protection scope of the disclosure.

Source of Biological Material

C57 control mice and db/db mice were purchased from Nanjing Model Animal Center (Nanjing, China). 293T cells were from China Center for Type Culture Collection in Wuhan University (Wuhan, China). Enzyme, plasmid, cells used in any of the following experiments and reagents, kit, consumable materials for routine use in various molecular biology experiments are commercially available.

The 1^{st} group of examples: The biomarker for diagnosing diabetes of the invention

This group of examples provides a biomarker for the diagnosing and/or early warning diabetes.

All embodiments in this group have following common features: the biomarker for the diagnosing and/or early warning diabetes comprises sequence fragment containing hsa-miR-320a; the hsa-miR-320a is shown as SEQ ID NO: 1.

In specific embodiments, the biomarker comprises: the hsa-miR-320a.

In a more specific embodiment, the biomarker is the hsa-miR-320a.

The 2^{nd} group of examples: A kit for diagnosing/early warning/risk assessing diabetes of this invention

This group of examples provides a kit for diagnosing and/or early warning diabetes. All examples in this group have following common features: the kit for diagnosing and/or early warning diabetes comprises reagent for quantitative detection of the biomarker of any of the 1^{st} group of examples.

In further examples, the kit comprises reagent for quantitative detection of hsa-miR-320a.

In specific examples, the reagent for quantitative detection of hsa-miR-320a comprises a specific primer pairs for hsa-miR-320a.

In more specific examples, the specific primer pairs are commercially-available MIRQ0000510-1-1.

In preferred examples, the regents for quantitative detection of hsa-miR-320a also comprises reverse transcription reagents; and/or reverse transcription PCR reagents;
preferably, the reverse transcription reagent comprises: RT Primer Mix, 2×TS reaction buffer, RNase free H₂O, TS enzyme;
further preferably, the reverse transcription PCR reagent comprises: 2×SYBR Green Mix, RNase free H₂O.

In some examples, the quantitative detection refers to fluorescent quantitative PCR detection;

In other preferred examples, the kit comprises miR-320a RT-primers, miR-320a real-time PCR forward primers, miR-320areal-time PCR reverse primers, SYBR Green I, TS reaction enzyme, TS reaction buffer and DEPC ddH₂O.

The 3^{rd} group of examples: Use of biomarker of this invention.

This group of examples provides use of the biomarkers described in any of the 1^{st} group of examples in preparing reagents for diagnosing diabetes.

The 4^{th} group of examples: a drug for preventing and treating diabetes of this invention.

This group of examples provides a drug for preventing and treating diabetes. All examples in this group have following common features: an active pharmaceutical ingredient of the drug is taking hsa-miR-320a as drug target, and efficacy of preventing and treating diabetes is achieved by binding, and/or capturing, and/or degrading hsa-miR-320a, and/or down-regulating expression of hsa-miR-320a; The hsa-miR-320a is shown as SEQ ID NO: 1.

In some examples, the active pharmaceutical ingredient of the drug comprises: substance that can degrade hsa-miR-320a and/or down-regulate expression of hsa-miR-320a; preferably, the active pharmaceutical ingredient of the drug is a substance that can down-regulate expression of the hsa-miR-320a.

In some other examples, the active pharmaceutical ingredient of the drug comprises: sequence fragment anti-hsa-miR-320a that is antisense complementary to hsa-miR-320a;

In preferred examples, the antisense complementary refers to that length of the anti-hsa-miR-320a reverse complementary to full-length or partial sequence of the hsa-miR-320a is a sequence fragment of 15-25 bases.

In other preferred examples, the active pharmaceutical ingredient of the drug is a recombinant plasmid expressing the sequence fragment anti-hsa-miR-320a that is antisense complementary to hsa-miR-320a.

In some specific examples, the active pharmaceutical ingredient of the drug is a recombinant adeno-associated virus plasmid pAAV-D(+)-anti-hsa-miR-320a, by which expressed sequence fragment anti-hsa-miR-320a is antisense complementary to hsa-miR-320a;

In some more specific examples, the pAAV-D(+)-anti-miR-320a is constructed by inserting fragment amplified through primer pairs with sequences shown as SEQ ID NO: 4 and SEQ ID NO: 5 into an adenovirus expression vector pAAV-D(+).

In further examples, the drug also comprises pharmaceutically acceptable excipients and/or reagents for buffering, culturing and/or amplifying the recombinant adeno-associated virus plasmid pAAV-D(+)-anti-miR-320a.

The 5^{th} group of examples: a method screening drug for preventing and treating diabetes of this invention

This group of examples provides a method screening drug for preventing and treating diabetes. All embodiments in this group have the following common features: detecting whether candidate substance can bind, capture and degrade hsa-miR-320a and/or down regulate expression;

In preferred examples, substance that can reduce expression level of hsa-mir-320a is screened.

The 6^{th} group of examples: a method for preparing a drug for preventing and treating diabetes of this invention. This group of examples provides a method for preparing a drug for preventing and treating diabetes. All examples in this group have the following common features: the method comprises: taking substance that degrade hsa-miR-320a or down-regulate expression of hsa-miR-320a; sequence fragment anti-hsa-miR-320a antisense complementary to hsa-miR-320a;
recombinant plasmid expressing a sequence fragment of anti-hsa-miR-320a antisense complementary to hsa-miR-320a; and/or,
a recombinant adeno-associated virus vector expressing a sequence fragment of anti-hsa-miR-320a antisense complementary to hsa-miR-320a as an active ingredient in antidiabetic drug.

In further examples, the method also comprises: primer pairs expressing sequence fragment anti-hsa-miR-320a antisense complementary to hsa-miR-320a is inserted into expression vector to prepare recombinant plasmid stably expressing sequence fragment anti-hsa-miR-320a antisense complementary to hsa-miR-320a.

In specific examples, primer pairs which can express the sequence fragment of anti-hsa-miR-320a antisense complementary to hsa-miR-320a are shown as SEQ ID NO: 4 and SEQ ID NO: 5; the expression vector is adeno-associated virus expression vector pAAV-D(+).

### Example 1

miRNA detection in peripheral blood of diabetic patients.

1. Peripheral blood was collected from 200 diabetic patients and 200 healthy controls. The samples were centrifuged at 3500 rpm for 6 min at room temperature, and the upper plasma was collected and stored at −80° C. 1 mL TRIZOL LS (Invitrogen, USA) was added into 0.25 ml peripheral blood plasma. RNA was extracted using RNasey Mini Kit.

The quality of RNA was detected by Nanodrop® ND-1000.

2. Expression of hsa-miR-320a was detected by real-time PCR through using miRNA detection kit from Guangzhou Ribo Company.

miRNA reverse transcription:

RT Primer Mix:

miRNA RT Primer 1 μL

U6 RT Primer 1 μL

RNase free H2O 78 μL

Reverse transcription reaction system:

RNA template 2 μg

RT Primer Mix 4 μL

RNase free H2O up to 19 μL

Above systems were mixed, centrifuged instantaneously, incubated at 70° C. for 10 min, ice incubated for 2 min; then the following reagents were added:

2×TS reaction buffer 25 μL

TS enzyme 2.5 μL

RNase free H2O 3.5 μL

Reverse transcription reaction procedure:

42° C. for 60 min, 70° C. for 10 min. Standby at 4° C. after shutdown, and the product was stored at −20° C.

miRNAs real-time PCR:

Reaction system: 2×SYBR Green Mix 9 μL

RT product 2 μL

miRNA Forward Primer 2 μL (purchased from Guangzhou Ribo Biotechnology Company Limited)

miRNA Reverse Primer 2 μL (purchased from Guangzhou Ribo Biotechnology Company Limited)

RNase-free H2O 5 μL

reaction procedure:

95° C. 30 sec—(95° C. 10 sec—60° C. 20 sec—70° C. 1 sec)×40 cycles—Melting Curve.

The results showed that expression level of hsa-miR-320a in peripheral blood of diabetic patients was increased (FIG. 1A). Fasting blood glucose were also increased significantly (FIG. 1B). Expression level of hsa-miR-320a was positively correlated with fasting blood glucose level (FIG. 1C). hsa-miR-320a could be used for diagnosing diabetes (FIG. 1D).

Sequence of hsa-miR-320a: aaaagcuggguugagagggcga (SEQ ID NO: 1).

### Example 2

Preparing kit to assess diabetes risk.

The kit comprises the following main components: a set of specific reverse transcription primers and real-time PCR primer pairs used for amplifying miR-320a; a set of specific reverse transcription primers and real-time PCR primer pairs used to amplify control RNA (U6) and related reagents. Components and contents are as follows (100 times) and stored at −20° C.:

| Components | Concentration | Volume | | Source |
|---|---|---|---|---|
| miR-320a RT primer | 5 μmol/L | 50 | μL | Guangzhou |
| | | | | Ribo Company |
| miR-320a real-time | 5 μmol/L | 200 | μL | Guangzhou |
| PCR forward primer | | | | Ribo Company |
| miR-320a real-time | 5 μmol/L | 200 | L | Guangzhou |
| PCR reverse primer | | | | Ribo Company |
| U6 reverse | 5 μmol/L | 50 | μL | Guangzhou |
| transcription primer | | | | Ribo Company |
| U6 real-time PCR | 5 μmol/L | 200 | μL | Guangzhou |
| forward primer | | | | Ribo Company |
| U6 real-time PCR | 5 μmol/L | 200 | μL | Guangzhou |
| reverse primer | | | | Ribo Company |
| SYBR Green I | 2× | 1 | mL | Life Company |
| TS reaction enzyme | 2× | 150 | μL | Life Company |
| TS reaction buffer | | 1.5 | mL | Life Company |
| DEPC ddH₂O | | 1 | mL | — |

The above reagents are provided by various companies and have been commercialized. Specific detection methods and related reaction parameters refer to Example 1.

### Example 3

Validation of accuracy of kit for early warning/diagnosing diabetes:

Currently, fasting blood glucose detection and OGTT test are commonly used to diagnose diabetes. Blood samples were collected from 100 patients who had been previously diagnosed with diabetes by above method. The biomarker provided by any of the 1^{st} group of examples and the kit for early warning/diagnosing diabetes provided by the 2^{nd} group of examples of the disclosure are used for molecular detection. The diagnostic criteria for test are: The expression of miR-320a was higher than?? (currently no large-scale population data) is diagnosed as diabetes. The final result shows that there are ??, of which expression level of miR-320a in blood samples of diabetic patients is higher than?. Accuracy of the kit for early warning/diagnosing diabetes is about ?%. (there is no large-scale population data at present).

Currently, fasting blood glucose detection and OGTT test are commonly used to diagnose diabetes. Blood samples were collected from 100 patients who had been previously diagnosed with diabetes by above method. The biomarker provided by any of the 1^{st} group of examples and the kit for early warning/diagnosing diabetes provided by the 2^{nd} group of examples of the disclosure are used for molecular detection. The diagnostic criteria for test are: The expression of miR-320a was higher than that of 50 nmol/L is diagnosed as diabetes. The final result shows that there are 83 patients of which expression level of miR-320a in blood samples of diabetic patients is higher than 50 nmol/L. Accuracy of the kit for early warning/diagnosing diabetes is about 83%.

### Example 4

Construction of recombinant adeno-associated virus

1. Synthesis of inserting sequence

Two reverse complementary strands of hsa-miR-320a and reverse complementary anti-hsa-miR-320a were designed and synthesized, respectively, based on the sequence of hsa-miR-320a (FIG. 2).

Primer pairs expressing hsa-miR-320a:

hsa-miR-320a-Sense:
(SEQ ID NO: 2)
5′ agctttcgccctctcaacccagcttttttcaagagaaaaagctggg
ttgagagggcgaccgc 3′
hsa-miR-320a-Antisense:
(SEQ ID NO: 3)
3′ aagcgggagagttgggtcgaaaaaagttctctttttcgacccaact
ctcccgctggcgccgg 5′
Primer pairs expressing hsa-miR-320a:
anti-hsa-miR-320a-Sense:
(SEQ ID NO: 4)
5′ agcttaaaagctgggttgagagggcgattcaagagatcgccctctc
aacccagcttttccgc 3′
anti-hsa-miR-320a-Antisense:
(SEQ ID NO: 5)
3′ attttcgacccaactctcccgctaagttctctagcgggagagttgg
gtcgaaaaggcgccgg 5′

2. Experiments carried out according to the system and temperature in manual Nuclease-Free Water 36 μL

Annealing Buffer for DNA Oligos (5×) 10 μL

DNA oligo A (50 M) 2 μL

DNA oligo B (50 M) 2 μL

90° C. for 3 min, 37° C. for 1 hr, stored at 4° C.

3. Enzyme digestion reaction

Eukaryotic expression vector pAAV-D (+) was digested by BamH I and Not I at 37° C. for 2 hr, digestion reaction system was as follows:

10×K Buffer 1 μL

BSA 1 μL

BamHI 1 μL

Not I 1 μL

pAAV-D(+) 2 μL

ddH₂O 14 μL

4. Agarose Gel electrophoresis recovery

Double digestion products were separated by 1% Agarose Gel electrophoresis, and then recovered by TaKaRa Agarose Gel DNA Purification Kit Ver. 2.0. Detailed procedure is as follows:
1). 1×TAE buffer agarose gel was prepared and agarose gel electrophoresis was performed to target DNA;
2). Agarose gel containing the target DNA was cut out under ultraviolet light;
3). Weigh the gel block, calculate the volume of the gel block, cutup the gel block;
4). Add DR-I Buffer with volume being 3 times as many as gel block volume into the glue block, and heat the glue block to be melt at 75° C.;
5). Add DR-II Buffer with volume being 1/2 as many as volume of DR-I Buffer to the glue block, and mix evenly; when separating DNA fragments less than 400 bp, isopropyl alcohol with a final concentration of 20% should be added to the solution.
6). Place the Spin Column on Collection Tube;
7). The solution in above step 5 was transferred to Spin Column, centrifuged at 12000 rpm for 1 min, and the filtrate was discarded;
8). 500 μL Rinse A was added into the Spin Column, centrifuged at 12000 rpm for 30 sec, and the filtrate was discarded;
9). 700 μL Rinse B was added into the Spin Column, centrifuged at 12000 rpm for 30 sec, and the filtrate was discarded;
10). Repeat above step 9;
11). The Spin Column was placed on the Collection Tube and centrifuged at 12000 rpm for 1 min;
12). The Spin Column was placed on a new 1.5 mL centrifuge tube, and 25 μL of 60° C. preheated Elution Buffer was added to the center of the Spin Column membrane, and stood for 1 min at room temperature;
13). Centrifuged at 12000 rpm for 1 min to elute DNA.

5. Plasmid ligation
1). The pAAV-D(+) vector and the synthesized DNA fragments were ligated by T4 ligase and incubated overnight at 16° C. as follows:

10× T4 DNA ligase buffer 2.5 μL

DNA 0.3 pmol

Vector 0.03 pmol

T4 DNA ligase 1 μL

ddH₂O up to 25 μL
2). Add whole volume (25 μL) to 100 μL DH5a receptor cells and place in ice for 30 min;
3). After heat for 45 sec at 42° C., and then place in ice for 1 min;
4). 500 μL antibiotic free LB medium was added, and the culture was shaken at 100 rpm at 37° C. for 60 min;
5). Culture on LB plate medium containing Amp+, then white monoclonal colonies were selected.
6. Plasmid Extraction at small scale

Select monoclonal colonies, add them to 3 mL amp+LB liquid medium, and shake at 37° C. 280 rpm overnight. The plasmid was extracted using EasyPure Plasmid MiniPrep Kit purchased from Beijing TransGen Biotechnology Company Limited. The specific steps were as follows:
1). Take 1.5 ml of overnight cultured bacteria, centrifuge at 10000 g for 1 min, and suck up supernatant as much as possible;
2). Add 250 μL colorless solution RB (containing RNase A), shake to suspense bacterial precipitation;
3). Add 250 μL blue solution LB, gently turn up and down and mix for 4-6 times to fully lyse the bacteria to form a blue transparent solution;
4). Add 350 μL yellow solution NB, gently mix for 5-6 times until a compact yellow agglutination block formed, and stand at room temperature for 2 min;
5). Centrifuge at 15000 g for 5 min, carefully absorb the supernatant and add it to the adsorption column;
6). Centrifuge at 15000 g for 1 min and discard the effluent;
7). Add 650 μL solution WB, centrifuge at 15000 g for 1 min, and discard the effluent;
8). Centrifuge at 15000 g for 2 min to completely remove the residual WB;
9). Place the adsorption column in a new EP tube and add 20 μL EB preheated at 70° C., standing at room temperature for 1 min;
10). Centrifuge at 10000 g for 1 min, elute DNA, and store the washed DNA at −20° C.
7. Plasmid identification

The constructed plasmids were identified by double enzyme digestion and sequencing, and the eukaryotic expression plasmids pAAV-D(+)-miR-320a and pAAV-D(+)-anti-miR-320a were obtained. Their structures are shown in FIG. 3.
8. Plasmid Extraction at large scale

Prepare 1 L sterile conical flask, add 300 ml sterile LB medium, and add ampicillin solution to the final concentration of 100 μg/ml. Add 50 μL of the required plasmid (pxx2, pxx8 or pxx9; phelper; pAAV-D(+), pAAV-D(+)-miR-320a or pAAV-D(+)-anti-miR-320a), 280 rpm, 37° C. overnight culture. Plasmids were extracted according to manual of E.Z.N.A.® Endo-Free Plasmid Maxi Kit purchased from OMEGA company. The specific steps were as follows:
1). Collect bacteria by being centrifuged at 5000 g at room temperature for 10 min;
2). Discard the culture medium, add 10 ml Solution I/RNase A mixture, vortex for oscillation and complete resuspension;
3). Add 10 ml Solution II into the resuspended mixture, gently reverse and mix it for 10-15 times, and place it at room temperature for 2 min;
4). Add 5 ml Buffer N3 and gently reverse it several times to form white flocculent precipitation;
5). Put HiBind column in the collection pipe, add 5 ml Buffer GPS, stand at room temperature for 3-10 min, centrifuge at 5000 g for 5 min, discard the filtrate, and put the column back into the collection pipe;
6). Pour the bacterial lysate into the syringe filter, stand for 2 min, insert and push the piston to collect the filtered lysate;
7). Add 1/10 volume of ETR into the filtered pyrolysis solution, reverse it for 7-10 times, and then ice bath for 10 min;
8). After water bath at 42° C. for 5 min, centrifuge at 5000 g at room temperature for 5 min, transfer the supernatant to a new centrifuge tube, add 0.5 times volume of absolute ethanol, mix well and stand at room temperature for 2 min;
9). Transfer the mixed solution to the activated HiBind column, centrifuge at room temperature of 5000 g for 5 min, and discard the filtrate;
10). Reinstall the binding column into the collection pipe, add 10 ml HB buffer, centrifuge at 5000 g at room temperature for 5 min, and discard the filtrate;
11). Reinstall the binding column into the collection pipe, add 15 ml DNA wash buffer, centrifuge at 5000 g at room temperature for 5 min, and discard the filtrate;
12). Repeat above step;
13). Discard the filtrate, reinstall the binding column into the collection pipe, and centrifuge at 6000 g for 15 min;
14). Take out the binding column and dry it at 65° C. for 10 min;
15). Put the binding column into a new centrifuge tube, add 1-3 ml Endotoxin free Elusion Buffer preheated at 70° C., stand at room temperature for 2 min, and then centrifuge at 6000 g for 5 min to elute DNA.
9. rAAV mediated virus packaging

293T cells (human embryonic renal epithelial cells) grow to 90%. 1-2 hours before calcium and phosphorus transfection, change 12-15 ml of fresh medium (including serum) for each Petri dish, first add calcium chloride (CaCl₂)) into 50 ml centrifuge tube, and then add plasmid to form Ca-DNA mixture, fully mix well, and slowly drop 2×HEBS BUFFER into Ca-DNA mixture to form Ca-DNA-P mixture, Shake the centrifuge tube while adding 2×HEBS to fully mix to form calcium and phosphorus particles. After 8-12 hours, change 18-20 ml serum-free medium. After 72 hours, suck and discard the medium, wash it with PBS for 3 times, add 1 ml Tris+NaCl (pH 8.5) to each Petri dish, curet the cells with a curette, collect them in a clean centrifuge tube and freeze at −80° C.
10. Virus purification

Take out the cells frozen at −80° C., thaw and dissolve at 37° C., freeze and thaw repeatedly for 4 times, centrifuge at 8000 g for 15 min, put the supernatant into a clean centrifuge tube, and discard the cell precipitation.

Fully mix anhydrous ethanol precooled at −20° C. with rAAV in the volume ratio of 3:1. After being placed in the refrigerator at −20° C. for 2 hours, centrifuge at 4° C. by 13000 rpm for 15 minutes, and discard the supernatant; After ethanol volatilization, add corresponding volume of Tris+NaCl (pH 8.5) to dissolve the precipitation. Filter with millipore filter (0.22 m).
11. Virus titer determination

Sample treatment: rAAV virus solution 40 μL

Protease K (20 mg/ml) 5 μL

55° C., reaction 1 hr;

Phenol: chloroform: isoamyl alcohol 45 μL

4° C., 12000 g, centrifugation for 5 min to recover the aqueous phase;

Chloroform 45 μL

4° C., centrifuged at 12000 g for 5 min to recover the aqueous phase.

Real-time PCR:

Primer 1 (10 m) 0.4 μL

Primer 2 (10 m) 0.4 μL

SYBR Green I Mix 10 μL

ddH₂O 8.2 μL

Template 1 μL
95° C. 30 sec—(95° C. 5 sec—60° C. 5 sec—72° C. 20 sec)×40 cycles—Melting Curve

12. Virus transfection efficiency

After the purified virus was transfected into 293T cells for 48 hours, the proportion of transfected cells was observed by fluorescence microscope, and the transfection efficiency was more than 90% (FIG. 4).

### Example 5

A recombinant adeno-associated virus of rAAV9 type expressing hsa-miR-320a/hsa-anti-miR-320a was used as an example to test its therapeutic effect on diabetes and complications.

1. Detection of blood glucose in db/db mice

12 weeks old C57 control and db/db diabetic mice were used to inject rAAV-miR-320a and rAAV-anti-miR-320a through caudal vein injection respectively. Titer of virus was 1×10¹¹ PFU. The fasting blood glucose of db/db mice was detected by taking caudal vein as sample to be tested through test paper at the end of the experiment (12 weeks later). The results showed that the blood glucose of db/db diabetic mice was significantly higher than that of C57 control mice. rAAV-miR-320a treatment significantly increased blood glucose, while rAAV-anti-miR-320a treatment significantly reduced blood glucose (FIG. 5).

2. Cardiac function detection of db/db mice

The cardiac function of db/db mice was measured by cardiac ultrasound at the end of the experiment. The methods were as follows:

The instrument used is an ultrasonic instrument equipped with a 30 MHz high-frequency probe. After the mice were anesthetized with isoflurane, the mice were placed on their back on detection platform, and two-dimensional images of the left ventricle were collected along the horizontal short axis and long axis of left ventricular papillary muscle near sternum of mice. At the same time, M-mode ultrasound images of more than 5 consecutive cardiac cycles were obtained respectively under guidance of two-dimensional images. According to the collected images, software was used to analyze results, and cardiac hemodynamic indexes detected by cardiac ultrasound were obtained. After analysis by relevant software, the following indexes were calculated: Heart Rate (HR); Left Ventricular Internal Dimension, diastole, LVIDd; Left Ventricular Internal Dimension, systole, LVIDs, Left Ventricular Posterior Wall, diastole, LVPWd; Left Ventricular Posterior Wall, systole, LVPWs; Interventricular septal thickness; diastole; IVSd; Interventricular septal thickness, systole, IVSs; Ejection Fraction, EF and Fractional Shortening, FS, and etc. The results showed that compared with C57 control mice, the cardiac systolic function of db/db mice was significantly impaired. rAAV-anti-miR-320a treatment could significantly improve the impaired cardiac function of db/db mice, while rAAV-miR-320a treatment could significantly aggravate the cardiac function of damaged db/db mice (FIG. 6).

### Example 6

Animal clinical treatment verification on drug preventing and treating diabetes of the invention

Hundreds of diabetic mice were treated with drug preventing and treating diabetes of this invention. Symptoms of each diabetic mouse before treatment include high blood sugar and diabetic complications, for example, impaired cardiac contractility.

Treatment of diabetes was specifically as follows: the drug preventing and treating diabetes provided by any of the 4^{th} group of examples, and/or, drug preventing and treating diabetes screened by the method provided by any of the 5^{th} group of examples, and/or, drug preventing and treating diabetes prepared by the method provided by any of the 6^{th} group of examples were injected into db/db mice once through caudal vein injection at their age of 12 weeks. Virus titer was 1.0×10¹¹ PFU/mouse, and blood glucose and cardiac contractility of each mouse were detected after 12 weeks. It was found that blood glucose and heart systolic function of all diabetic mice were significantly improved after the above treatment (comparison data charts before and after treatment in each mouse were similar to results shown in FIGS. 4 and 5. In order to save space, this disclosure does not list them one by one). This shows that accuracy rate of animal clinical treatment for drug preventing and treating diabetes of this invention is 100%. At the same time, all mice injected with above drugs were followed up and observed half a year later. There were no abnormalities or other side effects. At the same time, blood glucose remained stable, and symptoms of complications did not appear again within half a year.

1-20. (canceled)

21. A drug for preventing and treating diabetes, wherein an active pharmaceutical ingredient of the drug targets hsa-miR-320a, and efficacy of preventing and treating diabetes is achieved by binding, and/or capturing, and/or degrading hsa-miR-320a, and/or down-regulating expression of hsa-miR-320a; and the hsa-miR-320a is shown as SEQ ID NO: 1.

22. The drug of claim 21, wherein the active pharmaceutical ingredient of the drug comprises: a substance for degrading hsa-miR-320a and/or suppressing expression of hsa-miR-320a.

23. The drug of claim 22, wherein the active pharmaceutical ingredient of the drug comprises a sequence fragment anti-hsa-miR-320a that is antisense complementary to hsa-miR-320a; and
a sequence fragment in the anti-hsa-miR-320a has 15 to 25 bases reverse complementary to a full-length or partial sequence of the hsa-miR-320a.

24. The drug of claim 23, wherein the active pharmaceutical ingredient of the drug is a recombinant plasmid expressing the sequence fragment anti-hsa-miR-320a that is antisense complementary to hsa-miR-320a.

25. The drug of claim 24, wherein the active pharmaceutical ingredient of the drug is a recombinant adeno-associated virus plasmid pAAV-D(+)-anti-hsa-miR-320a, by which expressed sequence fragment anti-hsa-miR-320a is antisense complementary to hsa-miR-320a;
the pAAV-D(+)-anti-miR-320a is constructed by inserting a fragment amplified through primer pairs with sequences shown as SEQ ID NO: 4 and SEQ ID NO: 5 into an adenovirus expression vector pAAV-D(+).

26. The drug of claim 25, wherein the drug further comprises pharmaceutically acceptable excipients and/or reagents for buffering, culturing and/or amplifying the recombinant adeno-associated virus plasmid pAAV-D(+)-anti-miR-320a.

27. A method of screening a drug for preventing and treating diabetes, the method comprising detecting whether a candidate substance binds, captures, degrades hsa-miR-320a and/or suppresses expression of hsa-miR-320a; the candidate substance being capable of reducing expression level of hsa-miR-320a.

28. A method for preparing a drug for preventing and treating diabetes, the method comprising:
providing a substance for degrading hsa-miR-320a or for suppressing expression of hsa-miR-320a;
providing a sequence fragment anti-hsa-miR-320a antisense complementary to hsa-miR-320a;
providing a recombinant plasmid expressing a sequence fragment of anti-hsa-miR-320a antisense complementary to hsa-miR-320a; and/or, providing a recombinant adeno-associated virus vector expressing a sequence fragment of anti-hsa-miR-320a antisense complementary to hsa-miR-320a as an active ingredient in an antidiabetic drug.

29. The method of claim 28, further comprising: inserting primer pairs expressing the sequence fragment anti-hsa-miR-320a antisense complementary to hsa-miR-320a into an expression vector to prepare the recombinant plasmid expressing the sequence fragment anti-hsa-miR-320a antisense complementary to hsa-miR-320a.

30. The method of claim 29, wherein the primer pairs expressing the sequence fragment of anti-hsa-miR-320a antisense complementary to hsa-miR-320a are shown as SEQ ID NO: 4 and SEQ ID NO: 5; and
the expression vector is adeno-associated virus expression vector pAAV-D(+).

31. A biomarker for diagnosis and/or early warning of diabetes, the biomarker comprising a sequence fragment comprising hsa-miR-320a; and the hsa-miR-320a is shown as SEQ ID NO: 1.

32. The biomarker of claim 31, wherein the biomarker is the hsa-miR-320a.

33. A kit for diagnosis and/or early warning of diabetes, the kit comprising a reagent for quantitative detection of the biomarker of claim 31.

34. A kit for diagnosis and/or early warning of diabetes, the kit comprising a reagent for quantitative detection of hsa-miR-320a.

35. The kit of claim 34, wherein the reagent for quantitative detection of hsa-miR-320a comprises a specific primer pair for hsa-miR-320a.

36. The kit of claim 35, wherein the specific primer pair is commercially-available IRQ0000510-1-1.

37. The kit of claim 35, wherein the reagent for quantitative detection of hsa-miR-320a further comprises a reverse transcription reagent; and/or a reverse transcription PCR reagent;
the reverse transcription reagent comprising:
RT Primer Mix, 2×TS reaction buffer, RNase free H₂O, TS enzyme;
the reverse transcription PCR reagent comprises: 2×SYBR Green Mix, RNase free H₂O.

38. The kit of claim 33, wherein the quantitative detection refers to fluorescent quantitative PCR detection; and
the kit comprises miR-320a RT-primers, miR-320a real-time PCR forward primers, miR-320areal-time PCR reverse primers, SYBR Green I, TS reaction enzyme, TS reaction buffer and DEPC ddH₂O.


SEQUENCE LISTING (5 sequences)

SEQ ID NO: 1 - RNA, 22 nt, Artificial Sequence
  Fully synthetic
aaaagcuggguugagagggcga

SEQ ID NO: 2 - DNA, 62 bp, Artificial Sequence
  Fully synthetic
agctttcgccctctcaacccagcttttttcaagagaaaaagctgggttgagagggcgaccgc

SEQ ID NO: 3 - DNA, 62 bp, Artificial Sequence
  Fully synthetic
aagcgggagagttgggtcgaaaaaagttctctttttcgacccaactctcccgctggcgccgg

SEQ ID NO: 4 - DNA, 62 bp, Artificial Sequence
  Fully synthetic
agcttaaaagctgggttgagagggcgattcaagagatcgccctctcaacccagcttttccgc

SEQ ID NO: 5 - DNA, 62 bp, Artificial Sequence
  Fully synthetic
attttcgacccaactctcccgctaagttctctagcgggagagttgggtcgaaaaggcgccgg